(19) United States
(12) Patent Application Publication
(10) Pub. No.: US 2013/0034738 A1
(43) Pub. Date: Feb. 7, 2013

(54) USE OF N-ETHYL PYRROLIDONE IN THE PRODUCTION OF ELECTRODES FOR DOUBLE-LAYER CAPACITORS

(71) Applicants: Peter Pilgram, Ingolstadt (DE); Armin Modlinger, Kamenz (DE)

(72) Inventors: Peter Pilgram, Ingolstadt (DE); Armin Modlinger, Kamenz (DE)

(73) Assignee: Evonik Litarion GmbH, Kamenz (DE)

(21) Appl. No.: 13/515,873

(22) PCT Filed: Nov. 16, 2010
(86) PCT No.: PCT/EP2010/067552

§ 371 Date: Jul. 24, 2012

(30) Foreign Application Priority Data
Dec. 16, 2009 (DE) 10 2009 054 718.5

(51) Int. Cl. H01G 13/00; H01B 1/04; B32B 15/082; H01G 9/155; H01B 1/12; C07D 207/12; B82Y 30/00

(52) U.S. Cl. 428/421; 427/79; 252/500; 252/511; 548/543; 361/502; 977/773

(57) ABSTRACT

The invention relates to the use of N-ethyl pyrrolidone in the production of electrodes for double-layer capacitors.

### CROSS-REFERENCE TO RELATED APPLICATIONS

This application is a national stage application (under 35 U.S.C. §371) of PCT/EP2010/067552, filed Nov. 16, 2010, which claims benefit of German application 10 2009 054 718.5, filed Dec. 16, 2009.

### BACKGROUND OF THE INVENTION

The present invention relates to a process for coating an aluminum-containing carrier in the production of an electrode of a double-layer capacitor, comprising the steps of: providing a composition comprising at least one solvent and/or dispersion medium and additionally at least one polymeric binder, and coating the carrier with the composition.

Double-layer capacitors (or electrochemical double-layer capacitors—EDLCs, ultracapacitors or supercaps) are electrochemical energy stores. They comprise two identical electrodes, which are wetted by an electrolyte. When a voltage is applied to the electrodes, electrically charged particles such as electrons or ions with the respective polarity accumulate at the electrodes. In order to bring the electrodes close to one another to save space, the electrodes may be separated from one another by an ion-conducting separator, in order to avoid short-circuits.

The double-layer capacitors differ from accumulators (secondary batteries), which likewise belong to the family of electrochemical energy stores, in that the electrically charged particles are merely accumulated at the electrodes. Within accumulators, the ions are incorporated into the electrodes. Furthermore, accumulators have two electrodes made from different materials.

In the production of electrodes for double-layer capacitors, according to the prior art, coating compositions or dispersions (so-called electrode slurries) comprising active materials, conductivity additives and binders are coated onto conductive foils in wet-chemical processes. These dispersions are produced using systems based either on water or on organic solvents. In the case of water-based systems, the binder is dispersed, with spot binding between the particles.

The behavior is different in the systems based on organic solvents, in which the binder dissolves fully in the solvent and the binder envelops the particles. For the coating operation, complete dissolution of the binder in the overall dispersion has to be ensured. A particularly suitable organic solvent for the production of electrodes has been found to be N-methylpyrrolidone (NMP).

Typically, the quality of the coating composition is checked by measuring the viscosity of the dispersion or of the solution. It should be noted that the viscosity of the coating composition can alter over the course of several hours, such that the composition is not used directly after production. A further problem with NMP is also that it is classified as toxic (teratogenic). For reasons of health and safety at work and for environmental reasons, there is therefore a need to replace this NMP. A further need is to provide solvent-based systems for production of these dispersions which acquire less solvent or dispersant than is the case with NMP.

In the prior art, in the field of accumulators, first attempts are known to substitute the less objectionable NEP (N-ethylpyrrolidone) for NMP:

Thus US 2009/0123841 A1 discloses an active material dispersion which can be applied by ink-jet techniques to the electrode foil of a lithium ion accumulator, said dispersion comprising a PVDF binder dissolved in NEP. Geared to the ink-jet printing, the dispersion, at 6 to 10 mPas, is of comparatively low viscosity.

EP 1978056 A1 describes an NEP-containing binder for active materials of accumulators. Viscosities in the range from 1 to 10 000 mPas are reported.

### BRIEF SUMMARY OF THE INVENTION

In light of this prior art, the object on which the invention is based is that of specifying a process for producing electrodes for double-layer capacitors, of the generic type specified at the outset, whose dispersion media or solvents satisfy safety and environmental provisions and, furthermore, exhibit good storage stability or storage stability improved in relation to NMP. Furthermore, the active materials and additives of a double-layer capacitor are to be able to be applied with a smaller quantity of dispersion medium or solvent, thus permitting higher solids contents in the electrode slurry.

The object is achieved in that N-ethylpyrrolidone (NEP) is used as the solvent and/or dispersion medium.

The invention thus provides a process for coating an aluminum-containing carrier in the course of production of an electrode for a double-layer capacitor, comprising the steps of:

a) providing a composition comprising at least one solvent and/or dispersion medium and additionally at least one polymeric binder,

b) coating the carrier with the composition,

wherein

the solvent and/or dispersion medium is or comprises N-ethylpyrrolidone.

Surprisingly it is found that active materials of the double-layer capacitors that are dispersed in NEP exhibit significantly better processing properties than those dissolved in NMP: in spite of a high solids content, low shear rates are achieved, and so the electrode slurry is readily manageable. This is especially surprising in so far as the active materials of the double-layer capacitors (especially activated carbon) must in principle have a much greater specific surface area than is commonly possessed by the active materials of accumulators. The high specific surface area of the active materials, then, significantly increases the viscosity of the electrode slurry.

### A BRIEF DESCRIPTION OF THE FIGURES

FIG. 1 describes, in a graphic representation, the viscosity characteristics η of electrode slurries with a solids content of 50% at 20° C. as a function of shear rate γ. The solids content is composed of 91.5% by weight of graphite (d50=16.8 μm, BET surface area 2.5 m²/g), 8% PVDF (Solvay Solef 1013) and 0.5% carbon black (Timcal, Super P).

FIG. 2 describes, in a graphic representation, the viscosity characteristics η of 9.1% by weight PVDF homopolymer solutions (PVDF homopolymer: melt flow index, MFI 1,5-3.5 g/10 min) in NEP or NMP at 20° C. as a function of shear rate γ.

FIG. 3 shows different binder systems: a) water-based system, b) solvent-based system.

### A DETAILED DESCRIPTION OF THE INVENTION

The coating process according to the invention thus envisages, in its broadest possible application, coating of a carrier with a composition which comprises at least N-ethylpyrrolidone and a polymeric binder. Typically, the coating composition comprises, in addition to the N-ethylpyrrolidone as a solvent and/or dispersion medium and the polymeric binder, also at least one so-called active material and a conductivity additive. The carrier coated with the coating composition is subsequently used further to produce an electrode, and the electrode in turn is used for the production of a double-layer capacitor. The production of the electrode typically also comprises the step of drying the coated carrier. More particularly, the solvent and/or dispersion medium is removed to form a solid, conductive layer on the carrier which layer is “active” after completion of the electrical energy store. By virtue of its aluminum content, the carrier itself is electrically conductive. More detailed remarks will be made hereinafter with regard to the individual components used and different aspects of the invention.

The polymeric binder has the task of ensuring good adhesion, both within the layer and to the carrier. Particular preference is given to using polyvinylidene fluoride homopolymers (PVDF). The use of PVDF is desirable due to its electrochemical stability and because the swelling of PVDF in the electrolyte of the electrical energy store which is finished at a later stage is low. Suitable binders for the inventive application are, however, also different PVDF copolymers, Teflon, polyamides, polynitriles and others. Preferred polymeric binders may be selected from the group comprising polyvinylidene fluoride homopolymers (PVDF); polyvinylidene fluoride copolymers (PVDF copolymers), e.g. PVDF-hexafluoropropylene (PVDF-HFP), PVDF-tetrafluoroethylene (PVDF-TFE) and PVDF-chlorotetrafluoroethylene (PVDF-CTFE); mixtures of PVDF and PVDF copolymer(s); polytetrafluoroethylene (PTFE); polyvinyl chloride (PVC); polyvinyl fluoride (PVF); polychlorotrifluoroethylene (PCTFE); polychlorotrifluoroethylene-ethylene (ECTFE); polytetrafluoroethylene-ethylene (ETFE); polytetrafluoroethylene-hexafluoropropene (FEP); polymethyl methacrylate (PMMA); polyethylene oxide (PEO); polypropylene oxide (PPO); polypropylene (PP); polyethylene (PE); polyimide (PI); and styrene-butadiene rubber (SBR). It is also optionally possible to use mixtures of binders, for example mixtures of PVDF homopolymer and copolymer in any desired ratios, or the binders may be crosslinkable.

As mentioned above, a coating composition of the type described here comprises, in addition to the solvent and/or dispersion medium and the polymeric binder, also at least one so-called active material. “Active material” is understood here by the person skilled in the art in general terms to mean a material which enables the reversible capture and deposition of electrically charged particles such as ions or electrodes. In the finished and operable double-layer capacitor, it is then possible for a charge or discharge current to flow, according to the structure of the store, during the capture and deposition operation of the electrically charged particles.

The capture and deposition operations each take place at the electrode in the case of charging or discharging. Double-layer capacitors have two electrodes which are identical in terms of their active material, these electrodes differing only in their polarity. In the process according to the invention, the coating composition therefore typically additionally comprises an active material which enables the reversible capture and deposition of electrically charged particles and which is preferably selected from the group comprising graphite; amorphous carbons; activated carbon. These substances may also be utilized in mixed form as active material.

The capacity of the active material to capture electrically charged particles, and hence the capacitance of the double-layer capacitor, is determined substantially by the specific surface area and by the mean pore diameter of the active material. A specific surface area of the active material of between 1000 and 2000 m²/g has proven optimal. The mean pore diameter of the active material is preferably between 2 to 5 nm.

Typically, a coating composition of the type described here additionally comprises at least one conductivity additive.

The latter has the task of improving the electrical conductivity of the coating and thus the capture and deposition of the electrically charged particles. Particular preference is given to using carbon blacks as conductive materials. Carbon blacks are carbonaceous fine solids with usually spherical primary particles of size between 10 and 300 nm, determined by means of TEM analysis to ASTM D 3849, which agglomerate to catenated aggregates and in some cases to aggregated lumps. However, suitable conductive materials for the inventive use are also fine graphites with d50 between 1 μm and 8 μm, preferably with d50 between 2 μm and 6 μm, determined by means of laser light scattering. It is optionally also possible to use mixtures of conductive materials, for example mixtures of carbon blacks and graphites in any desired ratios. In addition, the conductivity additives used may be carbon fibers.

According to the invention, the carrier consists of an aluminum-containing and therefore conductive material web or comprises such a material as a laminate. The carrier is preferably a rolled or electrolytically deposited aluminum foil. Laminates comprising such foils as carriers are also conceivable. The carriers may also be porous materials, wovens, nonwovens or expanded metal composed of aluminum, or polymeric foils, generally perforated foils, porous carriers, or textile structures such as wovens or nonwovens, coated with aluminum.

The in the coating composition used in accordance with the invention typically comprises an N-ethylpyrrolidone content of 30 to 80% by weight, preferably 40 to 70% by weight, and, preferably, a polymeric binder content of 0.5 to 8% by weight, preferably 1.0 to 5.0% by weight, and/or an active material content of 20 to 70% by weight, preferably 30 to 60% by weight, and/or a conductivity additive content of 0 to 5% by weight, preferably 0.2 to 3% by weight, based in each case on the composition.

The composition provided should have a viscosity in the range from 1000 to 7000 mPas, preferably 2000 to 5000 mPas, at a shear rate of 112 s⁻¹, measured at 20° C. The viscosity values are determined in the context of the present invention with the aid of a rheometer (RS 600 model) from Thermo Haake GmbH, Karlsruhe with a plate/plate measuring apparatus with a diameter of 35 mm. The viscosities are detected at shear rates of 1 to 500 s⁻¹. The measurements are recorded with the RheoWin software.

The present invention also provides a coated carrier produced by the above-described process, provided that such a carrier is suitable for the production of an electrode for double-layer capacitors. Correspondingly produced electrodes are likewise encompassed by the present invention.

The present invention further provides a composition comprising, as a solvent and/or dispersion medium, at least N-ethylpyrrolidone, and additionally at least one polymeric binder, an active material which enables the capture and deposition of electrically charged particles, and optionally at least one conductivity additive. A preferred composition of this type comprises an N-ethylpyrrolidone content of 30 to 80% by weight, preferably 40 to 70% by weight, a polymeric binder content of 0.5 to 8% by weight, preferably 1.0 to 5.0% by weight, an active material content of 20 to 70% by weight, preferably 30 to 60% by weight, and optionally a conductivity additive content of 0 to 5% by weight, preferably 0.2 to 3% by weight, based in each case on the composition.

The use of N-ethylpyrrolidone in electrode production for double-layer capacitors and the use of N-ethylpyrrolidone for production of a composition which is used for the coating of a carrier in the production of an electrode of a double-layer capacitor are likewise covered by the present invention.

N-Ethylpyrrolidone is very similar to N-methylpyrrolidone in many of its chemical and physical properties. However, it has a higher boiling point and flash point (NMP: b.p. 202° C., f.p. 91° C.; NEP: b.p. 208-210° C., f.p. 93° C.), which has a certain advantage from the point of view of occupational and storage safety.

More particularly, an essential feature of the invention is additionally that the use of N-ethylpyrrolidone as a solvent and/or dispersant enables application of active materials and optionally additives to a carrier with a smaller amount of dispersion medium, i.e. achievement of higher solids contents in the composition than is possible with N-methylpyrrolidone as the dispersion medium.

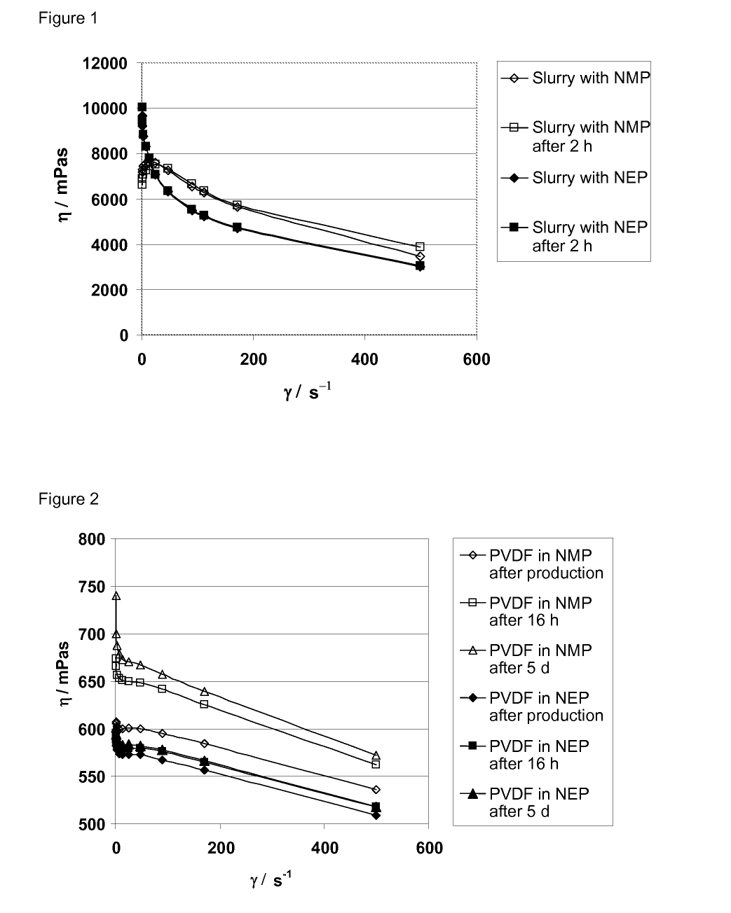

FIG. 1 describes, in a graphic representation, the viscosity characteristics η of electrode slurries with a solids content of 50% at 20° C. as a function of shear rate γ. The solids content is composed of 91.5% by weight of graphite (d50=16.8 μm, BET surface area 2.5 m²/g), 8% PVDF (Solvay Solef 1013) and 0.5% carbon black (Timcal, Super P).

FIG. 2 describes, in a graphic representation, the viscosity characteristics η of 9.1% by weight PVDF homopolymer solutions (PVDF homopolymer: melt flow index, MFI 1.5-3.5 g/10 min) in NEP or NMP at 20° C. as a function of shear rate γ.

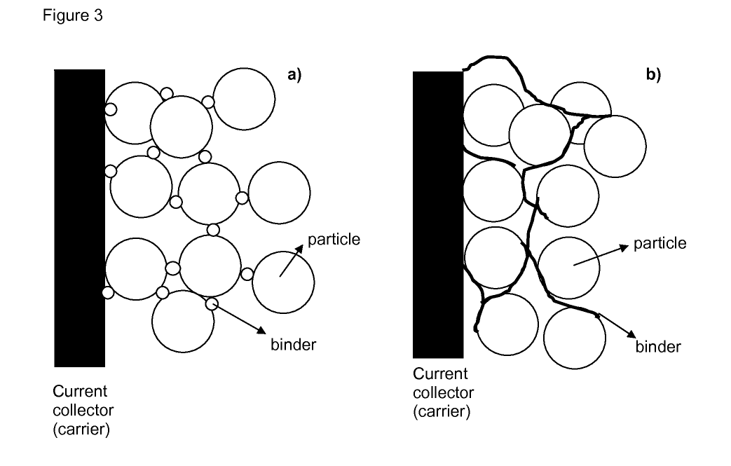

FIG. 3 shows different binder systems: a) water-based system, b) solvent-based system.

In the production of an inventive electrode slurry consisting of NMP or NEP, PVDF, graphite and a conductive carbon black, it was found that an NEP-based dispersion exhibits more significant lowering of viscosity with increasing shear rate than an NMP-based dispersion (FIG. 1). The crucial shear rates for typical coating processes are approx. 112 s⁻¹. Since NEP-based electrode slurries are present with lower viscosity at these shear rates, higher solids contents can be enabled in this case, thus achieving a reduction in the amount of dispersion medium. To produce such electrode slurries, the PVDF binder is frequently predissolved in the solvent in question. In the case of use of NEP as the solvent, much better storage stability is found compared to NMP as the solvent. A measure employed for the storage stability is the degree of increase in the viscosity in the solution in question with increasing storage time. The smaller the increase in viscosity with time, the greater the storage stability (FIG. 2).

### EXAMPLES

A 150 ml beaker was initially charged with the NMP or NEP, and the PVDF was added therein in portions within 15 min while stirring with a toothed disk (R1303 dissolver stirrer from IKA), diameter 42 mm, speed 750 rpm. At a PVDF content of 9.1% by weight (12.5 g in 125.0 g of solvent) the addition was stopped and stirring was continued for 1.5 h (750 rpm). Subsequently, the viscosity was determined as a function of time.

TABLE
Comparison of the solubility of PVDF in NEP and NMP

| | Viscosity in | | |
|---|---|---|---|
| Rheometer: HAAKE | mPas at shear rates of | | |
| RheoStress ® RS600 | 20 1/s | 73 1/s | 112 1/s |
| PVDF in NMP after preparation, | 600.4 | 597.1 | 592.3 |
| measurement 1 | | | |
| PVDF in NMP after preparation, | 601.5 | 600.5 | 596.1 |
| measurement 2 | | | |
| PVDF in NMP after 16 h, measurement 1 | 650.0 | 644.7 | 637.6 |
| PVDF in NMP after 16 h, measurement 2 | 651.2 | 647.4 | 641.1 |
| PVDF in NMP after 5 days, measurement 2 | 671.2 | 661.8 | 652.8 |
| PVDF in NMP after 27 days | 695.9 | 689.8 | 680.6 |
| PVDF in NEP after preparation, | 573.5 | 570.1 | 564.7 |
| measurement 1 | | | |
| PVDF in NEP after preparation, | 574.6 | 568.3 | 563.1 |
| measurement 2 | | | |
| PVDF in NEP after 16 h, measurement 1 | 579.1 | 577.8 | 573.3 |
| PVDF in NEP after 16 h, measurement 2 | 582.7 | 579.2 | 573.0 |
| PVDF in NEP after 5 days, measurement 2 | 583.0 | 580.2 | 575.3 |
| PVDF in NEP after 27 days | 584.6 | 578.6 | 572.1 |

It was found that, in the case of the solutions comprising NMP, the viscosity increased to a much greater degree in the course of time than was the case for the NEP solutions. It was also found that the NEP solution has a constant viscosity even after approx. 16 h while the viscosity continued to rise even after 5 days in the case of the NMP solution.

1-15. (canceled)

16. A process for coating an aluminum-containing carrier in the course of production of an electrode of a double-layer capacitor, comprising the steps of:
a) providing a composition comprising at least one solvent and/or dispersion medium and additionally at least one polymeric binder,
b) coating the carrier with the composition,
characterized in that
the solvent and/or dispersion medium is or comprises N-ethylpyrrolidone.

17. The process as claimed in claim 16, wherein the polymeric binder is selected from the group comprising polyvinylidene fluoride homopolymers (PVDF); polyvinylidene fluoride copolymers (PVDF copolymers); mixtures of PVDF and PVDF copolymer(s); polytetrafluoroethylene (PTFE); polyvinyl chloride (PVC); polyvinyl fluoride (PVF); polychlorotrifluoroethylene (PCTFE); polychlorotrifluoroethylene-ethylene (ECTFE); polytetrafluoroethylene-ethylene (ETFE); polytetrafluoroethylene-hexafluoropropene (FEP); polymethyl methacrylate (PMMA); polyethylene oxide (PEO); polypropylene oxide (PPO); polypropylene (PP); polyethylene (PE); polyimide (PI); and styrene-butadiene rubber (SBR).

18. The process as claimed in claim 16, wherein the polymeric binder is selected from the group comprising polyvinylidene fluoride homopolymers (PVDF); PVDF-hexafluoropropene (PVDF-HFP); PVDF-tetrafluoroethylene (PVDF-TFE); PVDF-chlorotetrafluoroethylene (PVDF-CTFE); mixtures of PVDF and PVDF copolymer(s); polytetrafluoroethylene (PTFE); polyvinyl chloride (PVC); polyvinyl fluoride (PVF); polychlorotrifluoroethylene (PCTFE); polychlorotrifluoroethylene-ethylene (ECTFE); polytetrafluoroethylene-ethylene (ETFE); polytetrafluoroethylene-hexafluoropropene (FEP); polymethyl methacrylate (PMMA); polyethylene oxide (PEO); polypropylene oxide (PPO); polypropylene (PP); polyethylene (PE); polyimide (PI); and styrene-butadiene rubber (SBR).

19. The process as claimed in claim 16, wherein the composition is a dispersion and additionally comprises an active material which enables the capture and deposition of electrically charged particles and which is selected from the group comprising graphite, amorphous carbons, activated carbon or mixtures thereof.

20. The process as claimed in claim 19, wherein the specific surface area of the active material is between 1000 and 2000 m²/g.

21. The process as claimed in claim 19, wherein the active material has a mean pore diameter of from 2 to 5 nm.

22. The process as claimed in claim 16, wherein the composition additionally comprises at least one conductivity additive which is selected from the group consisting of fine graphite with d50 between 1 μm and 8 μm; carbon blacks with primary particles between 10 and 80 nm; carbon fibers; and mixtures thereof.

23. The process as claimed in claim 16, wherein the carrier is an aluminum foil, a laminate comprising aluminum foil, an expanded aluminum metal, an aluminum-coated polymeric foil, or an aluminum-coated sheetlike textile structure.

24. The process as claimed in claim 16, wherein the carrier is an aluminum foil, a laminate comprising aluminum foil, an expanded aluminum metal, an aluminum-coated polymeric foil, or an aluminum-coated sheetlike nonwoven textile structure.

25. The process as claimed in claim 23, wherein the carrier is porous or regularly perforated.

26. The process as claimed in claim 16, wherein the composition comprises an N-ethylpyrrolidone content of 30 to 80% by weight, and an active material content of 20 to 70% by weight, and/or a conductivity additive content of 0 to 5% by weight, based in each case on the composition.

27. The process as claimed in claim 16, wherein the composition comprises an N-ethylpyrrolidone content of 30 to 80% by weight, and a polymeric binder content of 0.5 to 8% by weight, and/or an active material content of 20 to 70% by weight, and/or a conductivity additive content of 0 to 5% by weight, based in each case on the composition.

28. The process as claimed in claim 16, wherein the composition has a viscosity in the range from 1000 to 7000 mPas at a shear rate of 112 s⁻¹, measured at 20° C.

29. A coated carrier produced by the process according to claim 16.

30. A double-layer capacitor having at least one electrode, which is produced by the process as claimed in claim 16.

31. A composition comprising
at least one solvent and/or dispersion medium,
at least one polymeric binder,
an active material which enables the capture and deposition of electrically charged particles, and
optionally at least one conductivity additive,
wherein the solvent and/or dispersion medium is N-ethylpyrrolidone.

32. The composition as claimed in claim 31, wherein the active material is an activated carbon.

33. The composition as claimed in claim 31, wherein the N-ethylpyrrolidone content is from 30 to 80% by weight, the polymeric binder content is from 0.5 to 8% by weight, the active material content is from 20 to 70% by weight, and any conductivity additive content is from 0 to 5% by weight, based in each case on the composition.

34. A process for production of electrodes for double-layer capacitors which comprises utilizing N-ethylpyrrolidone.
